(19) United States
(12) Patent Application Publication
(10) Pub. No.: US 2001/0049459 A1
(43) Pub. Date: Dec. 6, 2001

(54) Test method for evaluating soluble polymer growth

(75) Inventor: Brigitte Benage, Wolcott, CT (US)

Correspondence Address: Kenneth D. Tremain, UNIROYAL CHEMICAL COMPANY, INC., World Headquarters, Middlebury, CT, 06749, US

(73) Assignee: CROMPTON CORPORATION

(21) Appl. No.: 09/760,539

(22) Filed: Jan. 16, 2001

Related U.S. Application Data
(60) Provisional application No. 60/185,167, filed on Feb. 25, 2000.

(51) Int. Cl. C07C 7/20; C08F 2/38

(52) U.S. Cl. 585/1; 252/380; 526/82

(57) ABSTRACT

The invention includes a method for evaluating the polymer growth inhibition ability of a compound or compounds. The invention involves adding a soluble monomeric, oligomeric, or polymeric seed capable of growing via a living polymerization mechanism to a liquid monomer under test conditions capable of causing a living polymer to grow via a living polymerization mechanism. Subjecting the same liquid monomer to the same test conditions then occurs in the absence of the soluble monomeric, oligomeric, or polymeric seed. Comparing the results of the two steps is then performed wherein the polymer growth inhibition ability of the compound or compounds is determined by the difference in the results. Another embodiment of the invention includes a method for evaluating the polymer growth inhibition by adding the soluble monomeric, oligomeric, or polymeric seed to the same liquid monomer solution under the same test conditions.

### BACKGROUND OF THE INVENTION

1. Field of the Invention

The present invention is directed to a test method for evaluating the effectiveness of various compounds in their ability to prevent polymer growth via a “living” polymerization mechanism when the “living” polymer is soluble in the monomer stream.

2. Description of Related Art

Many ethylenically unsaturated monomers undesirably polymerize at various stages of their manufacture, processing, handling, storage, and use. Polymerization, such as thermal polymerization, during their purification results in the loss of monomer, i.e., a lower yield, and an increase in the viscosity of any tars that may be produced. The processing and handling of the higher viscosity tars then requires higher temperature and work (energy cost) to remove residual monomer.

Polymerization can also result in equipment fouling, especially in the case of production of acrylic monomers. Such polymerization causes loss in production efficiency owing to the deposition of polymer in or on the equipment being used. These deposits must be removed from time to time, leading to additional loss in production of the monomer.

A wide variety of compounds has been proposed and used for inhibiting uncontrolled and undesired polymerization of ethylenically unsaturated monomers. However, many of these compounds have not been fully satisfactory. Accordingly, there has been a continuing need in the art for a testing means by which compositions intended for use as monomer polymerization inhibitors can be evaluated.

There are several mechanisms by which polymerization inhibitors work. One mode of action for polymerization inhibitors is for the inhibiting species to combine with the propagating polymer chain such that the polymerization of that polymer chain stops, i.e., a termination reaction. If such an inhibitor-terminated polymer chain is capable of participating in a dynamic equilibrium between a dormant species (the inhibitor-terminated chain) and an active polymer chain, it would be considered a “living” or quasiliving polymer. For example, Ivan, Macromol. Symp. 88:201-215 (1994) describes quasiliving polymerization as a polymerization in which “. . . only a portion of chain ends are active (propagating) and these are in equilibria with inactive (dormant, nonpropagating) chains . . . ” Shigemoto et al., Macromol. Rapid Commun. 17:347-351 (1996) state, “Well-defined polymers can be prepared by controlled/“living” radical polymerization in the presence of relatively stable radicals. These systems employ the principle of dynamic equilibration between dormant species and growing radicals via reversible homolytic cleavage of a covalent bond in dormant species.” Further, Greszta et al., Macromolecules 29:7661-7670 (1996) state, “The reversible homolytic cleavage of dormant species can be accomplished by either thermal, photochemical, or catalytic activation. The most successful approaches are as follows: homolytic cleavage of alkoxyamines and dithiocarbamates, use of various organometallic species, and catalyzed atom transfer radical polymerization.” Such a “living” polymer is capable of increasing in molecular weight (growing) through its reaction with additional monomer units of the same or different types of polymerizable monomers.

The method by which this “living” polymer grows is termed the “living” polymerization mechanism, and is depicted below.

M−Inh→M*+*Inh (1)

M*+*Inh→M−Inh (2)

M*+M′→M−M′* (3)

M−M′*+*Inh→M−M′−Inh (4)

Reactions (1) and (2) depict the dynamic equilibrium, with (2) being the termination reaction. Reaction (3) depicts growth of the polymer chain. Reaction (4) depicts re-termination of the growing polymer chain with the inhibiting species. The amount of growth over any period of time is dependent on the relative rate at which (2) occurs versus (3), as long as (1) occurs to some extent. The faster (2) is, relative to (3), the more time is needed for significant growth of the polymer. Under the conditions in which inhibitors are normally used, the concentration of the inhibiting species should be sufficiently high to cause reaction (2) to be much faster than reaction (3), otherwise it would not be an effective inhibiting system for commercial use. However, we have realized that even at an effective inhibiting amount of the inhibitor, growth can still occur, given sufficient time and temperature.

There are at least two scenarios in which “living” polymer can remain in a monomer purification train for an excessive amount of time.

First, the use of recycle can significantly increase the amount of time that the “living” polymer can remain in the purification train. To recycle unused inhibitor that is left in the purification stream after removal of the monomer, a portion of the residual stream is added to a feed stream earlier in the purification train. This residual stream typically contains inhibitor, small amounts of monomer, impurities in the monomer stream that have been concentrated by the purification process, and polymer formed during the production and purification process. Recycling this polymer will allow it time to grow if it is “living” polymer and the conditions of the purification train allow the “living” polymerization mechanism to occur. If this polymer grows via the “living” polymerization mechanism, excessive polymerization would cause loss in product yield, increased waste residues from the process, and potential plugging of equipment due to excessively high molecular weight polymer in the purification stream.

Second, occasionally, conditions in the plant/purification process can result in the formation of polymer within the purification train that is not dissolved by the monomer stream. If this polymer is caught in a dead space, or if it attaches to the metal on the inside of the equipment, it will not be washed out of the system. Thus, the polymer will remain within the system indefinitely (potentially for two or more years). If this polymer grows via the “living” polymerization mechanism, it could coat the inside of the equipment, causing inefficient separation of the monomer stream components and/or insufficient heating of the stream to enable purification. Such a situation would cause loss in product yield and could potentially cause an unscheduled shut-down of the plant in order to clean out the undissolved polymer in the equipment. Such a shut-down results in loss of monomer production and additional expense to clean out and dispose of the undissolved polymer.

### SUMMARY OF THE INVENTION

Given the potential loss in monomer yield as well as loss in monomer production and the additional economic drawbacks due to increased waste residues and cleaning of plugged equipment, a test method has now been developed to evaluate the effectiveness of various compounds in their ability to prevent polymer growth via a “living” polymerization mechanism when the “living” polymer is dissolved in the monomer stream.

More particularly, the present invention is directed to a method for evaluating the polymer growth inhibition ability of a compound or compounds comprising:

A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;

B) subjecting the same liquid monomer solution containing the compound or compounds of interest to the same test conditions as in A) in the absence of the soluble monomeric, oligomeric, or polymeric seed; and

C) comparing the results the results of A) with the results of B);

wherein the polymer growth inhibition ability of the compound or compounds is determined by the difference in the results obtained in A) and B).

In another embodiment, the present invention is directed to a method for evaluating the polymer growth inhibition ability of a compound or compounds comprising:

A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;

B) adding the soluble monomeric, oligomeric, or polymeric seed to the same liquid monomer solution under the same test conditions as in A) in the absence of the compound or compounds of interest; and

C) comparing the results the results of A) with the results of B);

wherein the polymer growth inhibition ability of the compound or compounds is determined by the difference in the results obtained in A) and B).

### DESCRIPTION OF THE PREFERRED EMBODIMENTS

As stated above, the present invention is directed to a method for evaluating the polymer growth inhibition ability of a compound or compounds comprising:

A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;

B) subjecting the same liquid monomer solution containing the compound or compounds of interest to the same test conditions as in A) in the absence of the soluble monomeric, oligomeric, or polymeric seed; and

C) comparing the results the results of A) with the results of B);

wherein the polymer growth inhibition ability of the compound or compounds is determined by the difference in the results obtained in A) and B).

Alternatively, the present invention is directed to a method for evaluating the polymer growth inhibition ability of a compound or compounds comprising:

A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;

B) adding the soluble monomeric, oligomeric, or polymeric seed to the same liquid monomer solution under the same test conditions as in A) in the absence of the compound or compounds of interest; and

C) comparing the results the results of A) with the results of B);

wherein the polymer growth inhibition ability of the compound or compounds is determined by the difference in the results obtained in A) and B).

The present invention thus comprises subjecting a soluble monomeric, oligomeric, or polymeric seed capable of growing via the “living” polymerization mechanism to a test method capable of evaluating the polymerization inhibition ability of a compound or blend of compounds under test conditions capable of causing the “living” polymer to grow via the “living” polymerization mechanism. Such test methods can include, but are not limited to, static tests, dynamic tests, small scale simulations of a distillation column and/or reboiler, and pilot units for a distillation train. The soluble seed can be of low molecular weight or high molecular weight, even polymeric in nature. There are various functional groups that can make the seed capable of growing, such as alkoxy amines, dithiocarbamates, organometallic species, and organohalides used in atom transfer radical polymerizations, as discussed by Greszta et al., in Macromolecules 29:7661-7670 (1996). If such a seed grows under test conditions capable of causing the “living” polymer to grow via the “living” polymerization mechanism, the data generated by that test should be different from the data obtained when no seed is present. For example, in a static test method for evaluation of polymerization inhibition ability, a rate of polymer formed over time can be obtained. That rate should increase if additional polymerization is occurring via the “living” polymerization mechanism. This could be demonstrated by a greater amount of polymer being formed over a specific time when the seed is present versus when it is not present. The compound or compounds being tested for their ability to prevent growth via a “living” polymerization mechanism would be effective growth inhibitors if a greater amount of polymer was not formed.

Another example would involve the use of a dynamic test method which is used to evaluate polymerization inhibition ability. In a dynamic test, the amount of polymer formed at steady state under a specific set of conditions indicates the effectiveness of the inhibiting system. The amount of polymer formed should increase if additional polymerization is occurring via the “living” polymerization mechanism. Thus, under a specific set of conditions, an increase in polymer formed at steady state in the test where the seed was added versus the test when the seed was absent indicates growth of the seed. This would indicate that the compound or compounds being tested were ineffective at preventing growth via a “living” polymerization mechanism.

In view of the many changes and modifications that can be made without departing from principles underlying the invention, reference should be made to the appended claims for an understanding of the scope of the protection to be afforded the invention.

What is claimed is:

1. A method for evaluating the polymer growth inhibition ability of a compound or compounds comprising:
A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;
B) subjecting the same liquid monomer solution containing the compound or compounds of interest to the same test conditions as in A) in the absence of the soluble monomeric, oligomeric, or polymeric seed; and
C) comparing the results the results of A) with the results of B); wherein the polymer growth inhibition ability of the compound or compounds is determined by the difference in the results obtained in A) and B).

2. The method of claim 1 wherein the evaluation is carried out by means of a static test, a dynamic test, a small scale simulation of a distillation column and/or reboiler, or a pilot unit for a distillation train.

3. The method of claim 1 wherein the seed contains an alkoxyamine functional group.

4. The method of claim 1 wherein the seed contains a dithiocarbamate functional group.

5. The method of claim 1 wherein the seed contains an organohalide functional group.

6. The method of claim 1 wherein the seed contains an organometallic species.

7. A method for evaluating the polymer growth inhibition ability of a compound or compounds comprising:
A) adding a soluble monomeric, oligomeric, or polymeric seed capable of growing via a living polymerization mechanism to a liquid monomer solution containing the compound or compounds of interest under test conditions capable of causing a living polymer to grow via a living polymerization mechanism;
B) adding the soluble monomeric, oligomeric, or polymeric seed to the same liquid monomer solution under the same test conditions as in A) in the absence of the compound or compounds of interest; and
C) comparing the results the results of A) with the results of B); wherein the polymer growth inhibition ability of the compound or compounds is determined by the difference in the results obtained in A) and B).

8. The method of claim 7 wherein the evaluation is carried out by means of a static test, a dynamic test, a small scale simulation of a distillation column and/or reboiler, or a pilot unit for a distillation train.

9. The method of claim 7 wherein the seed contains an alkoxyamine functional group.

10. The method of claim 7 wherein the seed contains a dithiocarbamate functional group.

11. The method of claim 7 wherein the seed contains an organohalide functional group.

12. The method of claim 7 wherein the seed contains an organometallic species.
